(19) United States
(12) Patent Application Publication
(10) Pub. No.: US 2018/0071045 A1
(43) Pub. Date: Mar. 15, 2018

(54) SEALED MEDICAL STERILIZATION CONTAINER USING NON-METALLIC FABRICATION MATERIALS OF CONSTRUCTION

(71) Applicant: INNOVATIVE STERILIZATION TECHNOLOGIES, LLC, Dayton, OH (US)

(72) Inventors: Scott COHEN, Centerville, OH (US); David Billman, Centerville, OH (US); Mike FAULKNER, Centerville, OH (US); Chuck KEMP, Huber Heights, OH (US); Gary WYGAL, Yellow Springs, OH (US)

(21) Appl. No.: 15/816,629

(22) Filed: Nov. 17, 2017

Related U.S. Application Data
(63) Continuation of application No. PCT/US2016/033070, filed on May 18, 2016.
(60) Provisional application No. 62/163,005, filed on May 18, 2015.

(51) Int. Cl. A61B 50/33; B65D 1/34; B65D 81/26

(52) U.S. Cl. CPC A61B 50/33; B65D 81/261; B65D 1/34

(57) ABSTRACT

A container (110) used for sterilization of medical components and utensils has a body (112) with a floor (114) and an upstanding wall (116). At least one vent (30), for entry of steam or the like, is formed in the floor. The vent has a plurality of vent holes, within a periphery defined by a ridge (34). A ledge (36) and a pair of raised portions (148) are provided inside the body to receive a rack on which the medical components are placed. A plurality of channels (144) in the floor of the body have a floor that is lower than the floor of the body, allowing gravity flow of liquid into the channels. The floor of each channel is adapted to enhance droplet formation, enhancing evaporation. The container is unitarily formed from a polymeric material selected for stability in the presence of high temperature and corrosive chemicals.

### CROSS-REFERENCE TO RELATED APPLICATIONS

This application is a bypass-continuation of, and makes a claim of priority to, PCT/US2016/033070, filed on 18 May 2016, which is a non-provisional application of, and makes a claim of priority to, U.S. provisional application 62/163,005, filed 18 May 2015. Both applications are incorporated by reference as if fully recited herein.

### TECHNICAL FIELD

This application relates a sterilization container, as is typically used to contain and sterilize medical components and utensils, where the container is manufactured from a polymeric material selected to be chemically unaffected by exposure to caustic environments. The container is structurally different from a sterilization container manufactured from stainless steel or aluminum.

### BACKGROUND

Sterilization containers, especially ridged sterilization containers are known in the art, where they are generally referred to in the medical industry as “trays.” They are used to sterilize components and utensils used in operating rooms and are manufactured from various materials, most commonly metals such as stainless steel or aluminum.

Because such trays, except as noted below, are all not considered truly sealed containers, extensive dry times are required in the sterilization cycle in order to obtain and maintain an adequate level of sterility. This dry time is required for all materials of construction, whether metal or non-metal. Drying of sterilized components and instruments in a non-water tight environment, whether utilizing porous disposable sterilization wrap material, or non-sealed containers of metal or non-metallic manufacture is required in order to eliminate any retained moisture in the set as such moisture can become a possible pathway for microbe migration from the exterior environment into these components causing contamination.

Accordingly, sterilization cycle time when using unsealed containers, (including/impacted by the above notes), is extensive regardless of the material of construction. This additional dry time can add up to two hours to the total sterilization processing time in order to adequately process a container. Additional process time outside of the sterilizer for the temperatures in the environment and container to slowly equalize (eliminate condensation created) is needed prior to usage in a sterile operating room environment.

In instances where non-metallic trays have been introduced to the marketplace as a general alternative to trays manufactured from metal, moisture retention and the additional extended drying times associated with such non-metallic trays beyond the already unacceptable times experienced with non-sealed metal trays have rendered such trays as being largely impractical commercially. Non-metallic containers tend to create a higher level of retained moisture through condensation versus metallic containers, due to the heat transfer driven by the temperature drop occasioned by the heat conductive properties of metal trays versus non-metallic containers substantially better insulating properties and thus their longer drying times upon removal from the sterilizer. These characteristics have precluded wide acceptance of non-metallic containers based on the increased risk of retained moisture being present when opened in a sterile environment. If moisture were to be present in a container, the occurrence is referenced as a “wet pack” and accordingly would be rejected by the surgical staff and sent for reprocessing.

One occasion in which non-metallic trays have been used is in the so-called “Immediate Use Steam Sterilization,” also referred to as “IUSS” or “Flash Sterilization.” In this situation, the drying step is unnecessary, since the sterilization is performed in the operating room environment and the tray is delivered directly from the sterilizer to the sterile environment for use. No shelf life is approved for containers processed by way of IUSS. However, such processing is not preferable and represents a compromise of medical safety only allowed due to the lack of a viable alternative to IUSS during surgical procedures. A well-known “plastic” IUSS container is commercially-available from Symmetry Surgical under the trademark FLASHPAK, although the product is not cleared to be placed on a shelf for storage of any length.

A further disadvantage of metal trays, and particularly aluminum and stainless steel trays is the inability of these trays to withstand a caustic (“high pH”) environment, particularly at pH levels above 12. Exposure to caustic solutions will destroy the metal trays in short order. Examples of situations requiring high pH sterilization include those involving the virus responsible for Ebola virus disease (EVD), various antibiotic-resistant organisms, and the agent responsible for Creutzfeldt-Jakob Disease (CJD). Treatment with a caustic solution is also required by law in certain geographic markets, without regard to hazardous waste implications.

The weight of sterilization containers is an issue confronted by surgical facilities. Governmental agencies such as the Occupational Safety and Health Administration (“OSHA”) and professional organizations such as the Association of peri-Operative Registered Nurses (AORN) require or strongly influence the allowable loaded weights of sterilization containers. Thus, the unloaded weight of the tray becomes an important factor. Use of a tray manufactured from a plastic or other non-metallic material potentially allows for a lighter tray thus providing loading of components and instruments to be sterilized within required weight tolerance as compared to similar sized trays manufactured from metals.

Depending on the alternative to metal material used and the manufacturing methodology applied, because of the expanded manufacturing alternatives available to such materials, non-metal material based framed containers are anticipated to also be less costly in their manufacture than similarly sized and functioning metal framed containers.

Additionally, use of plastic or similar non-metallic materials may also allow for clear tray tops allowing for viewing of the components and instruments contained therein, thus adding to operating room safety and assurance.

As a result of the above noted factors, current unsealed container sterilization technology is faced with two inadequate choices. The first choice is an unsealed metal container. It is likely to weigh above allowed regulatory limits, require extensive dry time of sterilized components and be unable to function in a high pH sterilization environment.

A second choice is an unsealed non-metallic container that will not cool quickly enough upon removal from an autoclave to avoid creating additional condensation. Even with extended dry times, longer “temperature acclamation” times are needed to slowly cool those non-metallic containers safely.

One alternative is a sealed container, such as those embodied in U.S. Pat. Nos. 6,319,481 and 7,595,032, both to Banks, which allow for a significantly reduced sterilization cycle time as compared to an unsealed sterilization container of whatever material of construction. These reductions in time occur because sealed containers create a sealed processing environment. Even when moisture remains after the sterilization cycle, such residual moisture does not allow pathogens a path of transference through the filters or between the component interfaces and remain sterile internally. These sealed containers do not require a dry time and as such require much less time to process the containers (all dry time including lead up/shut down is eliminated). Current sealed technologies utilize total process times of no more than 25-30 minutes and usually are completed in 20 minutes versus 2½+ hours for non-sealed containers. Sealed container processing, is a holistic system, and is achieved by way of the combination of the aforementioned tray design providing for a high velocity of airflow, specially (patent pending) designed filter covers which assure a water and particulate tight seal and utilization of specialty designed filters of an unusually high hydrostatic design and manufacture. This combination, when used in concert, allows for adequate airflow through the container assuring adequate heat and sterilization while also assuring that no water or particulate flow into the container occurs. Absent any of these three critical components, proper sealed sterilization having any shelf life does not result.

Sealed containers are currently only manufactured from aluminum metal, thus not allowing for the benefits noted above that would accrue from the use of plastics or other non-metallic materials having similar characteristics, i.e. among others, tolerance to high PH processing environments, lighter weight material than metals, improved visual access to the interior of the tray, were such alternatives to aluminum used in their construction. This is in spite of statements in the Banks patents that the inventive concept operates regardless of the material of construction.

Thus, the object is to provide a medical sterilization container that can be used in a caustic sterilization environment and which has a low weight per unit of volume contained, along with the reduced processing time afforded by sealed container sterilization.

### SUMMARY

This and other objects are achieved by the sterilization container for receiving a rack on which pieces to be sterilized are placed. Such a sterilization container has a body with a floor, an upstanding wall extending therefrom. At least one vent is formed in the floor of the body, comprising a plurality of vent holes, within a vent periphery defined by a ridge. A ledge and a pair of raised portions provided inside the body on the walls to receive the rack above the floor of the body. A plurality of channels are formed in the floor of the body, each channel having a floor that is lower than the floor of the body, thereby directing liquid into the channels. The floor of each channel is adapted to enhance droplet formation in liquid contained therein. The sterilization container is unitarily formed from a polymeric material selected for stability in the presence of high temperature and corrosive chemicals.

In many embodiments, the sterilization contain has a rectangular profile having two shorter sides and two longer sides, with the channels and the raised portions located along the longer sides. In such an embodiment, a pair of the channels is positioned along each longer side, adjacent to each of the raised portions.

In many embodiments, the surfaces inside the body, except for the floor of the channels, are rounded and smooth, to facilitate liquid flow. The surface of the floor of each of the channels is shaped or treated to “bead up” liquids thereon.

In some of the embodiments, the polymeric material is a polyphenyl sulfone. In other embodiments, the polymeric material is a poly ether ether ketone. In yet other embodiments, the polymeric material is a polyamide.

### BRIEF DESCRIPTION OF THE DRAWINGS

The various embodiments of the invention will be better understood when reference is made to the appended drawings, where identical parts are identified by identical reference numbers and wherein:

FIG. 1 shows a top perspective view of a sterilization container as typically known in the prior art; and

FIG. 2 is a top perspective view of a sterilization container as modified to incorporate the inventive concepts disclosed herein.

### DETAILED DESCRIPTION OF THE PREFERRED EMBODIMENTS

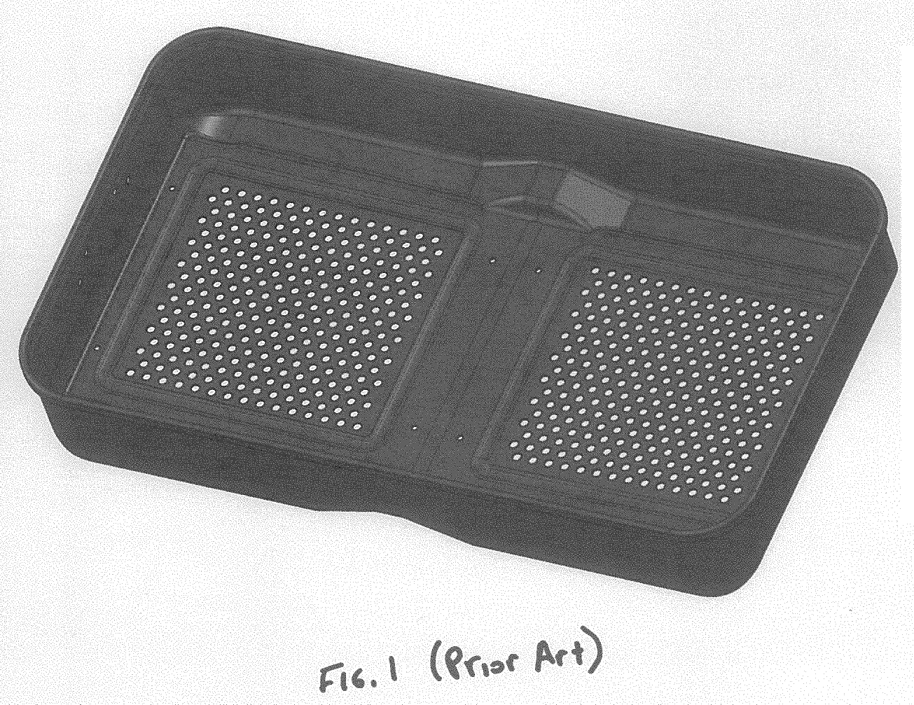

FIG. 1 shows, in top perspective view, an embodiment 10 of a sterilization container as known in the prior art. The embodiment 10 is one that would be molded from a polymeric material to withstand repeated exposures to caustic liquids. While resistant to the caustic liquids, the container 10 would have the disadvantages noted above with regard to higher amounts of retained condensation upon removal from a sterilization apparatus.

It is first noted that the container 10 is shown in an uncovered condition, for two reasons. First, the open condition allows the internal features of the container 10 to be readily observed. Second, it is highly desirable to be able to replace container 10 already in use with new containers embodying the inventive concept without the need to provide a new lid. It is to be understood that no inventive concepts, therefore, are included in the lid.

As shown, the container 10 has a body 12 that is preferably molded in a unitary manner. A floor 14 of the body 12 is surrounded by an upstanding wall 16 formed of wall segments. In a typical embodiment, the floor 14 will be rectangular, so the wall 16 will have two longer wall segments 18 that intersect the longer sides of the floor and two shorter wall segments 20 that intersect the shorter sides of the floor. The intersections of any pair of adjacent wall segments 18, 20 or of a wall segment 18 or 20 with the floor 14 are rounded, to minimize retention of liquids and to facilitate cleaning. In the depicted embodiment 10, the body has a pair of vents 30, each of which comprises a plurality of vent holes 32 and a surrounding ridge 34. These vents allow circulation of steam or the like during sterilization. A ledge 36 is positioned along each of the shorter wall segments 20 (only one is shown in FIG. 1, but the second is positioned along the opposite shorter wall segment). Correspondingly, a raised ramp portion 38 is formed along each of the longer wall segments 18, preferably near a midpoint of the length of the wall segment. As with the ledges 36, only one is depicted in FIG. 1. A ledge 40 on the top of each ramp portion 38 is arranged to interact with the ledges 36 to provide a series of surfaces for receiving a rack (not depicted), upon which the instruments being sterilized are placed.

Each raised ramp portion 38 also has a pair of inclined surfaces 42 that lead from the ledge 40 down to a channel 44. Viewed from the side, each of these channels 44 has a floor 46 that lies below the level of the vents 30, so that condensation or retained liquid will flow towards these channels and be retained in them.

The upper edges of the wall 16 are adapted to sealingly engage the lid and the body 12 may be provided with means that cooperates with a latching means on the lid, to secure the sealed engagement.

In the known prior art, all surfaces in the container, and particularly the inner surfaces, are smooth.

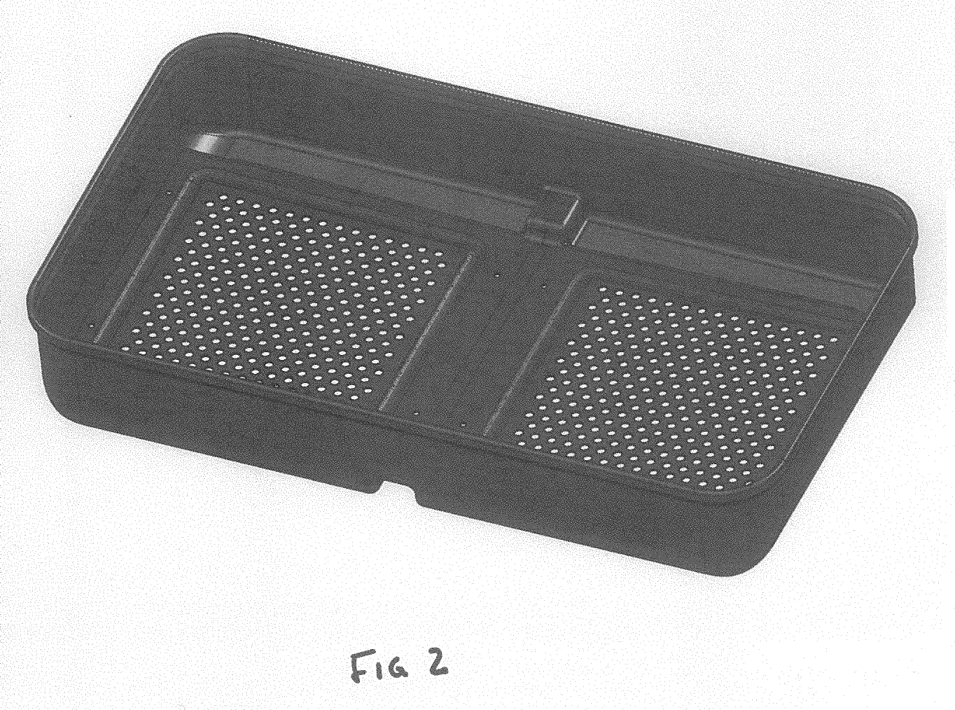

FIG. 2 shows, in a top perspective view similar to that in FIG. 1, an embodiment 110 of a sterilization container comprising the inventive concepts. The embodiment 110 is also molded from a polymeric material to withstand repeated exposures to caustic liquids.

As in FIG. 1, the embodiment 110 is shown in an uncovered condition to allow internal features to be readily observed. Also, it is a desirable feature of the container 110 to be useful with lids as known in the prior art.

Container 110 has a body 112, preferably molded in a unitary manner from a material that is resistant to caustic liquids. Particularly preferred materials that exhibit the desired physical and chemical properties would include: a polyphenyl sulfone, an example of which is commercially available from Solvay under the registered trademark RADEL; a poly ether ether ketone (“PEEK”), which is a commercially-available engineering plastic that is notable for its high melting point (above 600 F) and a polyamide material, such as a nylon, although a polyamide would probably need to be coated to be effectively used. A floor 114 of the body 112 is surrounded by an upstanding wall 116 formed of wall segments. In a typical embodiment, the floor 114 will be rectangular, so the wall 116 will have two longer wall segments 118 that intersect the longer sides of the floor and two shorter wall segments 120 that intersect the shorter sides of the floor. The intersections of any pair of adjacent wall segments 118, 120 or of a wall segment 118 or 120 with the floor 114 are rounded, to minimize retention of liquids and to facilitate cleaning. In the depicted embodiment 110, the body 112 has a pair of vents 30, each of which comprises a plurality of vent holes 32 and a surrounding ridge 34. These vents 30 allow circulation of steam or the like during sterilization. A ledge 36 is positioned along each of the shorter wall segments 120 (only one is shown in FIG. 2, but the second is positioned along the opposite shorter wall segment). Correspondingly, a raised portion 148 is formed along each of the longer wall segments 118, preferably near a midpoint of the length of the wall segment. As with the ledges 36, only one is depicted in FIG. 2. A ledge 152 on the top of each raised portion 148 is arranged to interact with the ledges 36 to provide a series of surfaces for receiving a rack (not depicted), upon which the instruments being sterilized are placed.

Each raised portion 148 is adjacent to a pair of channels 144. These channels 144 have a floor that is further below the level of the vents 30, thereby increasing the amount of condensation or retained liquid that will flow towards these channels and be retained in them. In way of further distinction, at least the floor of the channel 144 has a roughened or pebbled surface, and/or a surface that has been treated with a material that resists wetting by a polar liquid such as water. By doing this, water and aqueous solutions retained in the container 110, and especially in the channels 144, will tend to “bead up” rather than to spread out, due to the surface tension of the liquid. The resulting smaller collections of liquid allow for more rapid and thorough evaporation. Additionally, the surface area is increased allowing for heat transfer and dissipation of condensation. The combination of reducing the tendency of liquids, generally water, to cohere rapidly and increased surface area occasioned from a non-smooth surface, results in a more rapid evaporation of moistures created by condensation.

The upper edges of the wall 116 are adapted to sealingly engage the lid and the body 112 may be provided with means that cooperates with a latching means on the lid, to secure the sealed engagement.

The inventive concept is not to be limited by the disclosed embodiments, but is to be limited only by the enablement that the disclosure provides to those of skill in this art.

What is claimed is:

1. A sterilization container for receiving a rack on which pieces to be sterilized are placed, comprising:
a body, having a floor with an upstanding wall extending therefrom;
a vent formed in the floor, the vent comprising a plurality of vent holes within a periphery defined by a ridge;
a ledge and a pair of raised portions provided inside the body to receive the rack above the floor of the body; and
a plurality of channels, formed in the floor of the body, each channel having a floor that is lower than the floor of the body to direct liquid into the channels, the floor of each channel adapted to enhance droplet formation in liquid contained therein;
wherein the container is unitarily formed from a polymeric material selected for stability in the presence of high temperature and corrosive chemicals.

2. The sterilization container of claim 1 wherein the container has a rectangular profile having two shorter sides and two longer sides.

3. The sterilization container of claim 2, wherein the channels and the raised portions are located along the longer sides.

4. The sterilization container of claim 3, wherein a pair of the channels are positioned adjacent to each of the raised portions.

5. The sterilization container of claim 1 wherein the surfaces inside the body are rounded and smooth, except for the floor of the channels, to facilitate liquid flow.

6. The sterilization container of claim 5, wherein the surface of the floor of each of the channels is shaped or treated to “bead up” liquids thereon.

7. The sterilization container of claim 6, wherein the floor of each channel has a roughened surface.

8. The sterilization container of claim 6, wherein the floor of each channel is coated with a non-polar surfactant to cause beading of water.

9. The sterilization container of claim 1, wherein the polymeric material is a polyphenyl sulfone.

10. The sterilization container of claim 1, wherein the polymeric material is a poly ether ether ketone.

11. The sterilization container of claim 1, wherein the polymeric material is a polyamide.

12. The sterilization container of claim 4, wherein the polymeric material is a polyphenyl sulfone.

13. The sterilization container of claim 4, wherein the polymeric material is a poly ether ether ketone.

14. The sterilization container of claim 4, wherein the polymeric material is a polyamide.

15. The sterilization container of claim 4 wherein the surfaces inside the body are rounded and smooth, except for the floor of the channels, to facilitate liquid flow.

16. The sterilization container of claim 15, wherein the surface of the floor of each of the channels is shaped or treated to “bead up” liquids thereon.

17. The sterilization container of claim 16, wherein the polymeric material is a polyphenyl sulfone.

18. The sterilization container of claim 16, wherein the polymeric material is a poly ether ether ketone.

19. The sterilization container of claim 16, wherein the polymeric material is a polyamide.
